(19) United States
(12) Patent Application Publication
(10) Pub. No.: US 2005/0042199 A1
(43) Pub. Date: Feb. 24, 2005

(54) Fusion proteins containing tlp peptides

(76) Inventor: Giulio Tarro, Napoli (IT)

Correspondence Address: YOUNG & THOMPSON, 745 SOUTH 23RD STREET, 2ND FLOOR, ARLINGTON, VA, 22202, US

(21) Appl. No.: 10/497,178

(22) PCT Filed: Nov. 29, 2002
(86) PCT No.: PCT/EP02/13470

(30) Foreign Application Priority Data
Nov. 30, 2001 (IT) MI200A002527

(51) Int. Cl. A61K 39/20; C07K 14/54

(52) U.S. Cl. 424/85.2; 530/351

(57) ABSTRACT

Disclosed are fusion proteins obtained from a combination of TLP peptides and interleukin-2 (IL-2), and their use in cancer immunotherapy.

This invention relates to fusion proteins obtained from a combination of TLP peptides and interleukin-2 (IL-2), and their use in cancer immune therapy.

The abbreviation TLP (“Tumour Liberated Particles”) identifies protein complexes isolated from tumour tissues in accordance with the procedure initially described in EP 283433. Various peptides and proteins forming the TLP complex have been characterised. A 214 kDa TLP protein mainly expressed by lung cancer is described in Oncology, 1983 (40:248-253). Epitopes of the said protein and antibodies against them are described in WO98/01462. EP649433 describes a 100 kDa TLP protein isolated from lung cancer, and immunogenic peptides derived from it. These peptides have been used to generate monoclonal and polyclonal antibodies which are employed to characterise the TLP expression pattern in tissues of various origins and to validate TLP proteins as tumour antigens (WO94/01458 and WO98/15282).

Patent application MI2001A001380 describes a method for the preparation of TLP which allows its use in early tumour diagnosis.

Published patent WO01/62786 describes the isolation of cDNA that codes for a peptide of the TLP 100 kDa complex and its use in tumour immune therapy.

It has now been found that the immune response induced by the TLP peptides can be increased by their combination with an active fragment of interleukin 2 (IL-2). In particular it has been found that fusion proteins combining the amino acid sequences of TLP peptides and IL-2, elicit a cytotoxic response against tumour cells superior to that observed with TLP peptides alone.

The present invention therefore relates to a fusion protein or polypeptide obtained by combining the amino acid sequence of a TLP peptide with the amino acid sequence of an active fragment of IL-2.

The TLP peptides which can be used in accordance with the invention are described in WO 98/01462, WO 98/1458, WO 98/15282, EP 649433 and WO 01/62786, herein incorporated by reference in their entirety, and have the following amino acid sequences (the same peptide reference number will be used throughout the description):

| | |
|---|---|
| 1. | GPPEVQNAN |
| 2. | RTKNEASI |
| 3. | NQRNRD |
| 4. | GPPEVQNAN |
| 5. | TNKEASICPSVYLSTLPSIHSFTNSSIYYPCIHLSISLSVHPPLI |
| | HPSIYPSYSSIHSSSHHPCTHLSTHSVINPVKNFEHLLPIRPCTW |
| | TLG. |

Derivatives of the preceding peptides obtained by deletion, addition or substitution of one or more amino acids which retain the same immunogenic activity as the starting peptides are also included in the scope of the invention. “Active fragment of IL-2” means a region of the molecule comprising the first 90, preferably the first 100 amino acids starting from the amino terminal residue.

IL-2 may be of human or animal origin, and may be natural or recombinant. The human form whose sequence is deposited at GenBank accession number NP₁₃000577 is preferred. Moreover, it is possible to use variants of the IL-2 sequence (Cytokine (1997) 7:488-498) which retain its activity, e.g. which induce the growth of T-lymphocytes, regulate the activity of T-helper lymphocytes and stimulate cytotoxic T CD8 cells and NK cells (Theze et al., 1996, Immunol. Today, 17:481-486).

The term “fusion protein or polypeptide” in accordance with the invention means a (poly)peptide molecule constituted partly by the amino acid sequence of an active fragment of IL-2, the remainder being constituted by a TLP peptide sequence as specified above. Although the two sequences can be combined with any mutual orientation, it is preferable for the amino and carboxy-terminal domains to be occupied by IL-2 and the TLP peptide respectively. A linker with a sequence length which allows correct folding of the two sub-units can also be inserted between the two sequences. The linker will preferably comprise 10 to 30 neutral amino acids without hindering side chains, more preferably Gly and Ser residues.

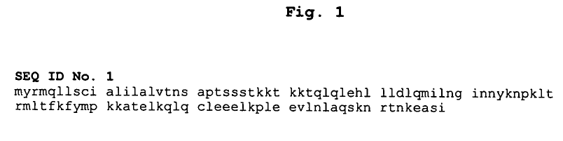

In accordance with a preferred embodiment, the fusion protein will contain a TLP peptide selected from 2 and 5 fused with an active fragment of IL-2. Even more preferred is the fusion protein having SEQ ID No. 1 (FIG. 1), wherein sequence 1-100 of IL-2 is fused with TLP peptide 2.

The two peptidic portions can be fused by chemical methods or recombinant DNA techniques. In this case, the cDNA that codes for IL-2 is fused “in frame” to the cDNA coding for the TLP peptide; after insertion of the recombinant cDNA in a suitable vector, the fusion protein is expressed in a eukaryote or prokaryote expression system. Alternatively, the cDNA that codes for the fusion product can be prepared with PCR using suitable amplification primers.

The fusion protein can also be prepared by synthesis in solution or solid phase (Merrifield, 1986, Science 232:341-347 and Barany and Merrifield, The Peptides, Gross and Meiehnhofer, eds N.Y. Academic Press, 1989, pp. 1-284), or with an automatic synthesiser (Stewart and Young, Solid Phase Peptide Synthesis, 2nd edition, Rockford Ill., Pierce Chemical Co., 1984).

The activity of the fusion proteins in accordance with the invention has been evaluated in cytotoxicity assays. Specifically, the activity of cytotoxic T-lymphocytes (CTL) against a syngenic tumour cell line naturally expressing human TLP and, respectively, the reactivity of an anti-human TLP immune rabbit serum against various human tumour lines have been tested. The details of the experiments are set out in the examples. The results clearly show that IL-2/TLP fusion proteins are more effective than single TLP peptides in inducing an antigen-specific cytotoxic immune response against tumour cells.

In accordance with another aspect, the invention therefore relates to the use of IL-2/TLP fusion polypeptides be the preparation a pharmaceutical composition useful in the prevention or treatment of tumours. The pharmaceutical compositions in accordance with the invention will contain an effective amount of fusion protein together with pharmaceutically acceptable vehicles and excipients. “Effective amount” means a quantity sufficient to activate the lymphocytes and trigger a cytotoxic response to the tumour. In accordance with a preferred embodiment, the said compositions are indicated for preventive vaccination of persons at risk of developing tumours or therapeutic vaccination of tumour patients. The techniques for preparation and use of vaccines are known to those skilled in the art, and are described, for example, in Paul, Fundamental Immunology, Raven Press, New York (1989) or Cryz, S. J., Immunotherapy and Vaccines, VCH Verlagsgesselshaft (1991). The vaccines are usually in the form of an injectable preparation, suspension or solution, but can also be made in the form of a solid or liposome-based preparation. The active constituents can be mixed with excipients such as emulsifying, buffering and adjuvant agents, thus increasing the efficacy of the vaccine.

The pharmaceutical compositions in accordance with the invention are particularly indicated for the prevention and treatment of tumours of the lung, colon/rectum, kidneys, bladder, testicles, ovaries, prostate, breast and cervix.

### EXAMPLE 1

### Preparation of Fusion Polypeptide IL2 (aa. 1-100)/TLP(aa. RTNKEASI)

Isolation and Cloning of cDNA

RT-PCR

The total RNA was extracted from various cell lines: A549 (lung cancer) HGC-27 (gastric cancer); DU-145 (prostate cancer) PA-1 (ovarian teratocarcinoma) HT29 (adenocarcinoma of colon) with RNAzol B reagent (TEL-TEST, Inc.) and reverse transcribed in 35 cycles (1 minute at 95° C., 2 minutes at 40° C. and 1 minute at 72° C.) using an oligonucleotide degenerated upstream (ACN AAY AAR GAR GCN TCN ATH TC, which corresponds to the amino acid sequence TNKEASI), and random hexamers as primers downstream. The products of PCR were subjected to electrophoresis on 1% agarose gel containing ethidium bromide. The PCR products were cloned in pGEM-T casy vector (Promega).

The resulting plasmid clones were sequenced by the chain termination method using an Applied Biosystems sequencer, model 373A. The open reading frame corresponding to SEQ ID No.1 was thus determined.

### EXAMPLE 2

### Cytotoxicity Test 1

BDIX rats syngenic to the BDH/K12 cell line which naturally express human TLP were used in this study. Line BDH/K12 is constituted by rat colon/rectum tumour cells.

The purpose of this experiment was to detect the production of cytotoxic T-lymphocytes (CTL) in BDIX rats experimentally, using a cytotoxicity test, after vaccination by inoculating a pre-determined dose of TLP/IL-2 in accordance with a conventional procedure.

Description of Method

The animals vaccinated with the test product produced cytotoxic lymphocytes (CTL) against the specific antigen.

The vaccinated animals were killed and the spleens containing CTL were removed.

Spleen cells (effector cells) of the vaccinated animals were co-cultured with radio-labelled target cells which express TAA on the surface. Cell lysis took place as a result of CTL/cell contact, with consequent release of radioactivity into the culture medium.

The radioactivity of the culture medium, detected with a γ-counter, is directly proportional to the activity of the CTLs, and therefore directly proportional to the vaccinogenic efficacy of the test substance.

Procedure

The test was based on 4 separate cycles, of immunisation. Each immunisation cycle was conducted on a group of four 8-week-old BDIX rats weighing approx. 250 g each. The product described in example 1 was used.
Day 0 (zero):
   rat 1: subcutaneous inoculation of 0.5 ml of saline (control)
   rat 2: subcutaneous inoculation of 0.5 ml of saline containing 70 μg of TLP/IL-2
   rat 3: subcutaneous inoculation of 0.5 ml of saline containing 70 μg of IL-2 (control)
   rat 4: subcutaneous inoculation of 0.5 ml of saline containing 70 μg of TLP peptide (control)

On the 14th day after the first immunisation, the rats were again inoculated by the same procedure (1st booster).

The subsequent boosters (2nd and 3rd) were performed on day 21 and 28 respectively. A week after the last immunisation (day 35) the animals were killed, and the spleens were removed and used to prepare the cytotoxicity test needed to detect the lytic activity of the specific anti-TLP T-lymphocytes (CTL), measuring the release of chromium 51 in the culture medium by DHD/K12 cells expressing TLP.

In the first stage of the test the DHD/K12 cells were counted and radio-labelled.

Then spleen cells (effector cells) originating from a previously vaccinated animal were counted and placed in contact with labelled target cells, in the ratios of 100:1, 50:1 and 25:1. The 10,000 DHD/K12 target cells were seeded in each culture.

Finally, the lytic activity with consequent release of chromium 51 was detected in the culture medium with a γ-counter.

The radioactivity measured represents the index of cytotoxicity (expressed as percentage toxicity) of the specific CTLs, taking the cytotoxicity measured in the test culture of rat 1 as 0 and the cytotoxicity used to radio-label the target cells cultured as 100.

The results of the experiment are set out below:

| | 100.1 | | 50.1 | | 25.1 | |
|---|---|---|---|---|---|---|
| | CYTOTOXI- | | CYTOTOXI- | | CYTOTOXI- | |
| | CITY | MEAN | CITY | MEAN | CITY | MEAN |
| CONTROL | | | | | | |
| SALINE | | | | | | |
| Rat 1 1st cycle | +1% | 1.25% | −1% | 1.00% | −1% | 1.00% |
| Rat 1 2nd cycle | +2% | | +1% | | +1% | |
| Rat 1 3rd cycle | +1% | | −1% | | −1% | |
| Rat 1 4th cycle | +1% | | +1% | | +1% | |
| VACCINE | | | | | | |
| D-TLP//IL2 | | | | | | |
| Rat 2 1st cycle | 78% | 76.50% | 83% | 77.00% | 75% | 73.25% |
| Rat 2 2nd cycle | 75% | | 72% | | 70% | |
| Rat 2 3rd cycle | 73% | | 74% | | 72% | |
| Rat 2 4th cycle | 80% | | 79% | | 76% | |
| CONTROL | | | | | | |
| IL2 | | | | | | |
| Rat 3 1st cycle | 2% | 4.5% | 2% | 1.75% | 1% | 1.00% |
| Rat 3 2nd cycle | 3% | | 2% | | 1% | |
| Rat 3 3rd cycle | 1% | | 2% | | 1% | |
| Rat 3 4th cycle | 3% | | 1% | | 1% | |
| CONTROL | | | | | | |
| D-TLP | | | | | | |
| Rat 4 1st cycle | 12% | 9.75% | 11% | 8.50% | 9% | 7.50% |
| Rat 4 2nd cycle | 8% | | 7% | | 6% | |
| Rat 4 3rd cycle | 9% | | 7% | | 8% | |
| Rat 4 4th cycle | 10% | | 9% | | 7% | |

### EXAMPLE 3

### Cytotoxicity Test 2

In this study, 2 rabbits were vaccinated with TLP/IL-2 antigen (Example 1) as follows:
day 0: a) a serum sample was taken from the animal (pre-immune serum)
   b) the first immunisation was performed by subcutaneous inoculation of a total of 0.6 ml of saline containing 0.5 mg of D.TLP//IL-2 at 6 points on the animal's back.
Day 14: 1st booster
Day21: 2nd booster
Day 28: 3rd booster
Day 35: Bleeding of animal from the ear vein and collection of immune serum.

The immune serum was characterised for TLP//IL-2 and TLP.

| Dilution | Dilution index | D-TLP//IL2 | D-TLP |
|---|---|---|---|
| 1 | 1/100 | 2.251 | 1.113 |
| 2 | 1/500 | 1.954 | 0.793 |
| 3 | 1/1000 | 1.021 | 0.658 |
| 4 | 1/2000 | 0.854 | 0.483 |
| 5 | 1/4000 | 0.425 | 0.322 |
| 6 | 1/8000 | 0.321 | 0.208 |
| 7 | 1/16000 | 0.212 | 0.131 |
| 8 | 1/32000 | 0.125 | 0.093 |
| | Diluent | 0.036 | 0.039 |
| | Peimm 1/10 | 0.102 | 0.098 |

The immune serum, prepared as described above, was then used in an in vitro cytotoxicity test on various human tumour cell lines.
Method:
1) seed 10 μl of decomplemented immune test serum in the well of a 96-well microdish
2) add the cells to 100 μl of complete medium (the number of cells seeded per well is 5000 for all lines except the Jurkat, in which case it is 35,000)
3) incubate the dish overnight at 37° C.
4) add 40 μl of MTT¹ to each well
5) incubate the dish at 37° C. for 4 hours
6) aspirate the supernatant and dissolve the granules with 100 μl of SDS² solution
7) incubate the dish for 1 h at 37° C.
8) read dish at 540 nm.
The percentage cytotoxicity is then calculated:

$$Cytotoxicity = \frac{OD of test sample - OD of blank}{O . D . of control cells - O . D . of blank}$$

Notes:
1) The MITT solution is made up as follows: 0.5 g of thiazolyl blue in 100 ml of PBS. Filter through 0.45 u and store in the dark at 4° C.
2) The SDS solution is made up as follows: 50 ml dimethylformamide+50 ml H₂O+20 g SDS under agitation on a heating plate. Should the pH exceed 7.4, buffer with a solution containing 49 ml of H₂O+1 ml acetic acid+0.125 ml of hydrochloric acid.

Results

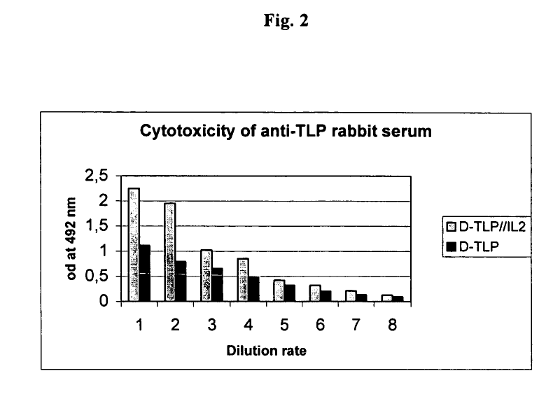

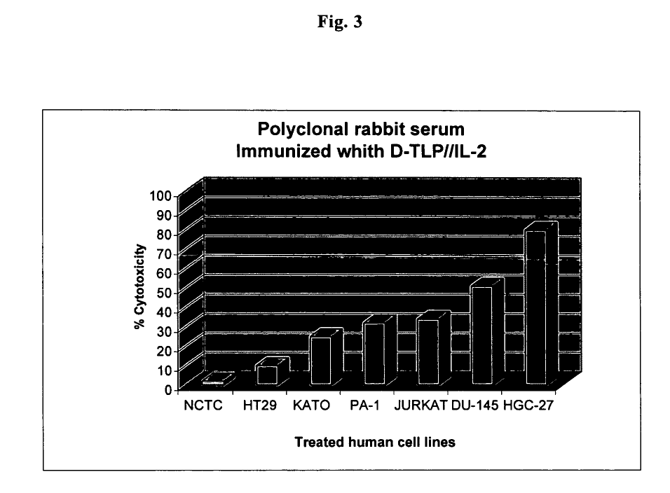

The results of the study are reported in the table below and in FIGS. 2 and 3.

| CELL LINE | % Citotox |
|---|---|
| NCTC (normal keratinocytes) | 2 |
| HT29 (adenocarcinoma of the colon) | 10 |
| KATO (adenocarcinoma of the stomach) | 25 |
| PA-1 (ovarian teratocarcinoma) | 32 |
| JURKAT (leukaemia T cells) | 34 |
| DU-145 (prostate cancer) | 51 |
| HGC-27 (gastric cancer) | 80 |

1. A fusion protein between an IL-2 fragment comprising the first N-terminal 90 amino acids and a TLP peptide selected from the group consisting of:
GPPEVQNAN RTKNEASI NQRNRD GPPEVQNAN
TNKEASICPSVYLSTLPSIHSFTNSSIYYPCIHL
SISLSVHPPLIHPSIYPSYSSIHSSSHHPCTHLS
THSVINPVKNFEHLLPIRPCTWTLG.

2. A fusion protein as claimed in claim 1, in which said IL-2 fragment contains amino acids 1-100 of the human IL-2 sequence.

3. A fusion protein as claimed in claim 2, wherein said IL-2 fragment is fused to the TLP peptide of sequence RTNKEASI.

4. A fusion protein as claimed in claim 1, wherein the IL-2 fragment and the TLP peptide are at the N-terminal and carboxy-terminal ends, respectively.

5. A fusion protein as claimed in claim 3, with sequence SEQ ID No. 1.

6. A fusion protein as claimed in claim 1, wherein the fragment IL-2 and the TLP peptide are separated by a spacer of 10 to 30 amino acids.

7. A fusion protein as claimed in claim 6, wherein said amino acids are selected from Gly and Ser.

8. A pharmaceutical composition containing an effective amount of a fusion protein as claimed in claim 1.

9. A pharmaceutical composition as claimed in claim 8, in the form of a vaccine.

10. A composition as claimed in claim 8, for use in the prevention or treatment of tumours of the lung, colon/rectum, kidneys, bladder, testicles, ovaries, prostate, breast and cervix.
